(19) United States
(12) Patent Application Publication
(10) Pub. No.: US 2024/0424199 A1
(43) Pub. Date: Dec. 26, 2024

(54) INTELLIGENT BRACELET CAPABLE OF INJECTING MEDICATIONS

(71) Applicant: Zimu WANG, Beijing (CN)

(72) Inventor: Zimu WANG, Beijing (CN)

(21) Appl. No.: 18/364,550

(22) Filed: Aug. 3, 2023

(30) Foreign Application Priority Data
Jun. 21, 2023 (CN) 202310744875.1

(51) Int. Cl. A61M 5/142

(52) U.S. Cl. CPC A61M 5/14244; A61M 2005/14272; A61M 2205/18; A61M 2205/502; A61M 2230/06; A61M 2230/205; A61M 2230/62; A61M 2230/63

(57) ABSTRACT

An intelligent bracelet capable of injecting medications is provided by the present application, including a multi-modal sensor assembly configured to acquire physical data of a wearer, a main control chip configured to detect abnormal physical conditions by adopting a machine learning method according to the physical data, a medication injection device configured to inject medications to the wearer when the abnormal physical conditions are detected, and a bracelet display interface configured to display the physical data and corresponding danger levels. An injection system of micro-electro-mechanical system (MEMS) is incorporated on the basis of the functions of the existing intelligent bracelet to automatically detect the physical indicators and abnormal state of the wearer, and to automatically inject adrenaline medications and give an alarm at the same time.

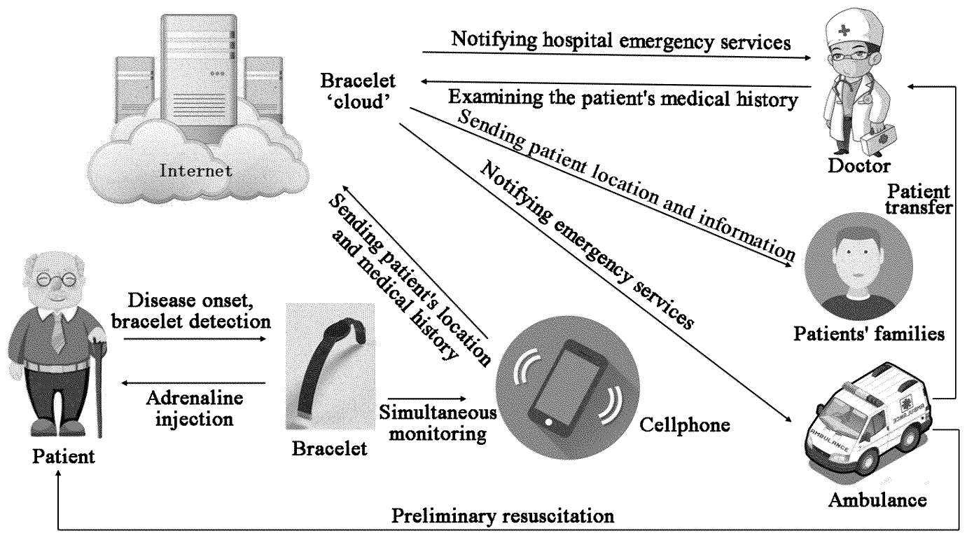

### CROSS-REFERENCE TO RELATED APPLICATIONS

This application claims priority to Chinese Patent Application No. 202310744875.1, filed on Jun. 21, 2023, the contents of which are hereby incorporated by reference.

### TECHNICAL FIELD

The present application belongs to the technical field of intelligent bracelet design, and in particular to an intelligent bracelet capable of injecting medications.

### BACKGROUND

Recent years see a growing number of people aged over 70 suffering from cardiovascular diseases as the global population ages, and it has been found in research that the vast majority of these people live alone most of the time and have companionship occasionally with their children on holidays. Most senior citizens have difficulty in operating smart devices due to blurred vision and shaky hands. Usually once feeling unwell, old people take first-aid medications, such as quick-acting heart pills or sublingual nitroglycerin, by oral administration and then seek further first aids. However, self-rescue options such as calling an emergency service or calling out for help to those around them are not possible when old people suffer sudden cardiovascular syncope. It is difficult for people to save themselves from a cardiovascular attack and the probability of being found and helped by others is low, and doctors find great difficulties in taking rescue measures, resulting in untimely and ineffective rescue.

### SUMMARY

It is an objective of the present application to provide an intelligent bracelet capable of injecting medications in order to solve the above-mentioned problems of the prior art.

To achieve the above objective, the present application provides an intelligent bracelet capable of injecting medications, including a multi-modal sensor assembly, a main control chip, a medication injection device and a bracelet display interface;

the multi-modal sensor assembly, the medication injection device and the bracelet display interface are connected with the main control chip;

the multi-modal sensor assembly is configured to acquire physical data of a wearer;

the main control chip includes a processor, a memory, a communication interface and a digital interface, and the main control chip is configured to detect abnormal physical conditions by adopting a machine learning method according to the physical data;

the medication injection device is configured to inject medications to the wearer when the abnormal physical conditions are detected; and

the bracelet display interface is configured to display the physical data and corresponding danger levels.

Optionally, the multi-modal sensor assembly further includes an inertial measurement unit (IMU), and the inertial measurement unit is configured to identify a fall of a human body, and the physical data includes heart rate and oxyhemoglobin saturation.

Optionally, the IMU adopts a fall detection algorithm model of long short-term memory (LSTM) for fall identification, including steps like sensor data acquiring, data preprocessing, feature extracting, threshold detecting, machine learning classifying, fall judging, posture analyzing, and warning and responding.

Optionally, early warning measures are activated when the multi-modal sensor assembly detects that a heart rate of the wearer is below 40 or above 160 and the oxyhemoglobin saturation is below 92% within 30 seconds, and a fall is detected from the wearer in the meantime.

Optionally, the early warning measures include: the bracelet display interface automatically pops up a display of a countdown for 30 seconds and simultaneously makes a sound of an alarm; if the wearer does not cancel the countdown by himself, the medication injection device is activated and the wearer is given an intramuscular injection of adrenaline; and

if the wearer cancels the countdown by himself, no medication injection is carried out, and a log of this event is recorded and the bracelet resumes to a normal state.

Optionally, the main control chip includes fall detection software, and the fall detection software adopts a pre-trained deep learning algorithm to optimize the fall detection algorithm model for performance in terms of a correct detection rate and a false alarm rate; and

a method of the pre-trained deep learning algorithm includes principal component analysis (PCA), linear discriminant analysis (LDA), over-sampling, transfer learning, online learning, model compression technology, cost function or decision threshold adjustment, data set fine adjustment, sensor sampling rate optimization, data processing and feature extraction pipeline.

Optionally, a home page of the bracelet display interface displays a current heart rate and a blood oxygen concentration of the wearer, with different colors indicating different physical danger levels; and

the danger levels include the highest danger, secondary danger and no danger, which are displayed in red, orange and green respectively.

Optionally, when the wearer is in the secondary danger, the bracelet display interface pops up an “SOS” interface for the wearer to determine whether to report an alarm; when the wearer is in a state of the highest danger, the medication injection device is automatically triggered to inject the medications.

The application has the technical effects that:

based on the functions of the existing intelligent bracelet, the present application incorporates the design of an injection system of micro-electro-mechanical system (MEMS), enabling the device to automatically detect the wearer's physical indicators and abnormal states, and to automatically inject epinephrine medications and automatically give an alarm at the same time, which helps improve the efficiency of the current cardiovascular disease emergency aid and develop a more effective medication delivery system; further, the present application enables the automatic injection of emergency medications and the simultaneous automatic alarm to save lives as well as medical and health resources.

### BRIEF DESCRIPTION OF THE DRAWING

The accompanying drawings which form part of this application are used to provide a further understanding of this application and the schematic embodiments of this application and the description thereof are used to explain this application and do not constitute an improper limitation of this application.

The FIGURE is a process illustrating a specific application of an intelligent bracelet capable of injecting medications provided by an embodiment of the present application.

### DETAILED DESCRIPTION OF THE EMBODIMENTS

It should be noted that the embodiments in this application and the features in the embodiments may be combined with each other without conflict. The present application is described in detail with reference to the drawings and embodiments.

It is noted that the steps illustrated in the process diagram of the accompanying drawings are executable in a computer system such as a set of computer-executable instructions and, although a logical sequence is illustrated in the process diagram, in some instances the steps shown or described may be executed in an order different from that shown herein.

### Embodiment 1

As shown in the FIGURE, an intelligent bracelet capable of injecting medications is provided in this embodiment, including a multi-modal sensor assembly, a main control chip, a medication injection device and a bracelet display interface; among them:

the multi-modal sensor assembly is configured to acquire physical data of a wearer;

the main control chip includes a processor, a memory, a communication interface and a digital interface, and the main control chip is configured to detect abnormal physical conditions are by adopting a machine learning method according to the physical data;

the medication injection device is configured to inject medications to the wearer when abnormal physical conditions are detected; and

the bracelet display interface is configured to display the physical data and corresponding danger levels.

The hardware and software modules and sensors in a system of the intelligent bracelet are required to be connected and controlled via the main control chip to achieve an overall functionality of the system.

The main control chip is the core of the intelligent bracelet system and is responsible for controlling and managing the various hardware modules and sensors, as well as processing and storing data. The main control chip usually integrates functions of a processor, a memory and a communication interface and is able to communicate with other hardware modules and sensors through various digital interfaces.

Various hardware modules are connected to a software system (including detection data and control signals. Typical digital interfaces include Inter-Integrated Circuit (I2C), Serial Peripheral Interface (SPI), and Universal Asynchronous Receiver/Transmitter (UART), etc.

Medication injection device-MEMS is an executive hardware module connected with the main control chip through digital interface.

The bracelet display interface, typically a liquid crystal display (LCD), is controlled by a display controller integrated with the main control chip.

The intelligent bracelet is embedded with a multi-modal physiological indicator abnormality and fall detection software as a core, which uses a machine learning algorithm model that can process heterogeneous source data from the multi-modal sensor assembly of the bracelet, relying on a pre-trained deep learning model to automatically detect patient physiological indicator abnormalities and fall postures in only a few fall samples, and give instructions to inject medications after autonomously determining that the patient is in imminent danger. It is configured for two different scenarios.

The multi-modal sensor assembly detects that:

the heart rate of the wearer is below 40 or above 160;

the oxyhemoglobin saturation of the wearer is below 92% (of a dangerous blood oxygen range); and

the inertial measurement unit (IMU) of the intelligent bracelet detects “falling”, that is, the body suddenly leans or falls.

If all three of these indicators are detected within 30 seconds, the bracelet interface automatically pops up a 30-second countdown display and gives an alarm at the same time. If the wearer does not cancel the countdown by himself, the sensor on the intelligent bracelet automatically sends a signal to activate the medication injection device for an intramuscular injection of adrenaline; and

if the wearer cancels it by himself, the software of the intelligent bracelet gives no instruction to inject the medications and a log of this event is recorded and the bracelet returns to its normal state.

The fall detection algorithm model of long short-term memory (LSTM) of the intelligent bracelet, which is fine-tuned and optimized with a small number of samples, distinguishes a fall from a normal physical movement of the patient, with following working steps:
1) heterogeneous sensor data collecting: sensors of IMU unit, including accelerometers, and magnetometers, continuously collects physical posture data; gyroscopes photoplethysmography (PPG) sensor collects physiological data of a heart rate and oxyhemoglobin saturation of the patient;
2) data preprocessing: the sensor data is pre-processed by denoising, filtering and normalization operations;
3) feature extracting: features associated with fall events, such as peak acceleration, angular velocity, etc., are extracted from the pre-processed data;
4) threshold detecting: the extracted feature values are examined for exceeding predefined thresholds, such as acceleration thresholds or angular velocity thresholds;
5) machine learning classifying: the extracted features are input into the trained model for classification;
6) fall judging: according to the results of threshold detection and/or machine learning classification, whether a fall event has occurred is judged;
7) posture analyzing: if a fall is detected, the posture (e.g. lying, sitting or standing) of the user after the fall is analyzed to further confirm the fall event; and
8) warning and responding: if a fall is confirmed, an alarm is triggered and the appropriate operation is performed, such as notifying an emergency contact or calling for help.

Considering the small sample amount of fallen body posture data from elderly patients in the real scenarios, the detection model uses pre-trained deep learning algorithms to improve the performance of the model in the following aspects, including key indicators such as a correct detection rate and a false alarm rate.
1) establishing a physical fall data set pre-trained model of open source;
2) feature selecting and dimensionality reducing: not all features extracted from the sensor data are equally useful for fall detection; some of them may be redundant or irrelevant, which may have a negative impact on the performance of the model; the model uses feature selection techniques, such as mutual information, correlation analysis or recursive feature elimination, to identify the most informative features; and dimensionality reduction methods such as principal component analysis (PCA) or linear discriminant analysis (LDA) can also reduce the feature space while retaining essential information;
3) unbalanced data processing: falling events are usually rare compared to normal activity, which may lead to an unbalanced dataset, and training models on such data may result in biased classifiers that are biased towards the majority of classes; to address this, the model uses resampling techniques such as oversampling a minority of classes (e.g. synthetic minority oversampling technique, or SMOTE) or undersampling a majority of classes (e.g. Tomek link, and neighbourhood cleaning rule); alternatively, the model's cost function or decision threshold is adjusted to account for class imbalance;
4) transfer learning: transfer learning is a technique where pre-trained model is fine-tuned for a specific task using a smaller dataset; this is particularly useful for fall detection models on bracelet devices, where collecting large annotated datasets can be challenging; the algorithm starts with a pre-trained deep learning model (e.g. a convolutional neural network (CNN) or LSTM trained on a relevant task) and fine-tunes it using a fall detection dataset; in this case, the convergence rate is increased with improved performance;
5) online learning: the algorithms for online learning update the model incrementally as new data becomes available, without having to retrain the entire model from scratch; this is useful in fall detection applications, where user behaviour and environmental conditions change over time; by adapting the model to these changes, a high level of detection accuracy is maintained;
6) energy and resource efficiency: intelligent bracelets often have limited computing resources and battery life; optimizing models and their implementation to ensure energy and resource efficiency is critical, and model compression techniques, such as pruning, quantization and knowledge distillation, allow for a reduction in model size and complexity without a significant reduction in accuracy; moreover, the sensor sampling rate, data processing and feature extraction pipeline are optimized to minimize energy consumption; and
7) evaluating indicators: accuracy may not be the best indicator to assess the performance of a fall detection model, especially when the dataset is unbalanced; it is critical to select appropriate evaluation indicators, such as accuracy, recall, f1 score or area under the receiver operating characteristic curve (ROC); these measures provide a more comprehensive understanding of model performance and help identify areas to be improved.

In summary, improving fall detection models on wearable devices requires a combination of strategies, including careful data pre-processing, feature engineering, model selection, performance optimization and user-centred design, and by implementing these techniques and continuously evaluating models in practical scenarios, more accurate and reliable fall detection systems can be constructed.

An example of LSTM model for fall detection process:
1) data collection
the following dependency libraries have been installed: pip install tensorflow numpy pandas scikit-learn;
sensor data such as accelerometer and gyroscope are collected and stored in a comma-separated values (CSV) file; the data shall include time stamps, accelerometer data (ax, ay, az) and gyroscope data (gx, gy, gz); a label column is also required to indicate whether the action at each time stamp is a fall (1 for a fall, 0 for normal activity);
2) data preprocessing
firstly, the data needs to be loaded and pre-processed; pre-processing the data usually includes removing missing values, normalizing the data, etc.; in this implementation, the ‘pandas’ library is used to load the data and the ‘scikit-learn’ library is used to normalize the data; the specific steps are as follows:
loading data;
removing missing values;
classifying data into input features and labels;
normalizing the data;
3) model construction
next, a simple LSTM model is constructed using ‘tensorflow’;
4) model training
the data is divided into a training set and a test set, and the model is then trained using the training set;
a time step dimension is added to LSTM;
the model is trained; and
5) model evaluation
the test set is used to evaluate model performance.
Interaction between patient and the intelligent bracelet

The home screen of the bracelet displays the wearer's current heart rate and oxyhemoglobin concentration, with different colors indicating the current danger level of the body (red for the highest danger 03, orange for the secondary danger 02 and green for the no danger 01). If the wearer feels unwell, or is in danger level 02, it is up to the wearer to decide if an alarm is required (the wearer needs to slide up the screen to pop up the “SOS” screen, but it does not activate the automatic injection process. The medication injection device is only activated automatically under state 03.

What has been described above represents only the specific embodiments of the present application, but the scope of protection of the present application is not limited to it. Any variation or substitution that may be readily thought of by any person skilled in the art within the technical scope disclosed by the present application should be covered by the scope of protection of the present application. Therefore, the scope of protection of this application should be subject to the scope of protection of the claims.

What is claimed is:

1. An intelligent bracelet capable of injecting medications, comprising a multi-modal sensor assembly, a main control chip, a medication injection device and a bracelet display interface;
wherein the multi-modal sensor assembly, the medication injection device and the bracelet display interface are connected with the main control chip;
the multi-modal sensor assembly is used to acquire physical data of a wearer;
the main control chip comprises a processor, a memory, a communication interface and a digital interface, and the main control chip is used to detect abnormal physical conditions by adopting a machine learning method according to the physical data;
the medication injection device is used to inject medications to the wearer when the abnormal physical conditions are detected; and
the bracelet display interface is used to display the physical data and corresponding danger levels.

2. The intelligent bracelet capable of injecting medications according to claim 1, wherein the multi-modal sensor assembly further comprises an inertial measurement unit, and the inertial measurement unit is used to identify a fall of a human body, and the physical data comprises a heart rate and oxyhemoglobin saturation.

3. The intelligent bracelet capable of injecting medications according to claim 2, wherein the inertial measurement unit adopts a fall detection algorithm model of long short-term memory (LSTM) for fall identification, and steps comprise sensor data acquiring, data preprocessing, feature extracting, threshold detecting, machine learning classifying, fall judging, posture analyzing, and warning and responding.

4. The intelligent bracelet capable of injecting medications according to claim 1, wherein an early warning measure is activated when the multi-modal sensor assembly detects a heart rate of the wearer below 40 or above 160 and the oxyhemoglobin saturation below 92% within 30 seconds together with a fall detected from the wearer.

5. The intelligent bracelet capable of injecting medications according to claim 4, wherein the early warning measure comprises: the bracelet display interface automatically popping up a display of a countdown for 30 seconds and simultaneously giving an alarm; if the wearer does not cancel the countdown, the medication injection device is activated and the wearer is given an intramuscular injection of adrenaline; and
if the wearer cancels the countdown, no medication injection is carried out, and a log of the event is recorded and the bracelet returns to a normal state.

6. The intelligent bracelet capable of injecting medications according to claim 1, wherein the main control chip comprises fall detection software, and the fall detection software adopts a pre-trained deep learning algorithm to optimize the fall detection algorithm model for performance in terms of a correct detection rate and a false alarm rate; and
a method of the pre-trained deep learning algorithm comprises principal component analysis (PCA), linear discriminant analysis (LDA), over-sampling, transfer learning, online learning, model compression technology, cost function or decision threshold adjustment, dataset fine adjustment, sensor sampling rate optimization, data processing and feature extraction pipeline.

7. The intelligent bracelet capable of injecting medications according to claim 1, wherein a home page of the bracelet display interface displays a current heart rate and a blood oxygen concentration of the wearer, with different colors indicating danger levels of the human body currently; and
the danger levels comprise a highest danger, a secondary danger and no danger and are displayed in red, orange and green respectively.

8. The intelligent bracelet capable of injecting medications according to claim 1, wherein when the wearer is in the secondary danger, the bracelet display interface pops up an “SOS” interface for the wearer to determine whether to report an alarm; when the wearer is in a state of the highest danger, the medication injection device is automatically activated to inject the medications.
